(19) United States
(12) Patent Application Publication
(10) Pub. No.: US 2002/0010128 A1
(43) Pub. Date: Jan. 24, 2002

(54) Treatment of hyperproliferative, inflammatory and related mucocutaneous disorders using inhibitors of mevalonate synthesis and metabolism

(76) Inventors: Thomas P. Parks, San Mateo, CA (US); Stephen Grayson, San Rafael, CA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/833,384

(22) Filed: Apr. 11, 2001

Related U.S. Application Data
(60) Provisional application No. 60/197,357, filed on Apr. 13, 2000.

(51) Int. Cl. A61K 38/00; A61K 31/56; A61K 31/40; A61K 31/366; A61K 31/045; A61K 31/235

(52) U.S. Cl. 514/2; 514/178; 514/423; 514/460; 514/533; 514/729

(57) ABSTRACT

The present invention provides methods for treating a variety of hyperproliferative and inflammatory mucocutaneous disorders, including, basal cell carcinoma, squamous cell carcinoma, psoriasis and atopic dermatitis, as well as skin irritation and disorders associated with skin aging and skin photodamage using inhibitors of cholesterol metabolism. The present invention further relates to the discovery that the combined use of several inhibitors of cholesterol metabolism produces synergistic effects. Furthermore, the present invention is directed to the use of inhibitors of cholesterol metabolism as excipients to enhance the effects of antiinflammatory drugs.

### CROSS-REFERENCES TO RELATED APPLICATIONS

This application claims the benefit of U.S. Provisional Patent Application Serial No. 60/197,357, filed Apr. 13, 2000, which is incorporated herein by reference in its entirety for all purposes.

### BACKGROUND OF THE INVENTION

Hyperproliferative and inflammatory mucocutaneous disorders affect millions of individuals in the United States every year. Such disorders range from mild to life threatening, and include, for example, skin cancer, atopic dermatitis, psoriasis, and asthma due to the inflammation of the lung mucosa. In addition, extrinsic skin aging can be caused by chronic inflammation and insufficient repair due to repetitive exposures to environmental insults, e.g., ultraviolet radiation. Aging of skin and in particular extrinsic aging can lead to any of a number of skin conditions requiring treatment. While certain treatments have been developed for some of these conditions, the treatments are often ineffective, not tolerated by certain individuals, or associated with one or more side effects that limit their use. With some conditions, no effective treatments exist whatsoever. Clearly, new treatments for hyperproliferative and inflammatory skin disorders are needed.

Eczema, also called eczematous dermatitis, is one example of a common inflammatory mucocutaneous disorder. Eczema is a red, itchy, noncontagious inflammation of the skin that may be acute or chronic, with red skin patches, pimples, crusts, or scabs occurring either alone or in combination. The skin may be dry, or it may discharge a watery fluid, resulting in an itching or burning sensation. The affected skin may become infected. The various causes of eczematous dermatitis are classified as either external (irritations, allergic reactions, exposure to certain microorganisms or chemicals, etc.), congenital (inherited predisposition) and environmental (stree, heat, etc.). Eczema may clear for years, only to reappear later at a different site. Eczema can come in any of several forms, including, most commonly, atopic dermatitis. Atopic dermatitis is very common in all parts of the world. This chronically relapsing inflammatory skin disorder affects about ten percent of infants and three percent of the U.S. population overall. The disease can occur at any age, but is most common in infants to young adults (see, Hanifin, J M et al, Arch. Dermatol, 135(12):1551 (1999), the teachings of which are incorporated herein by reference for all purposes). The face is often affected first, then the hands and feet. Sometimes dry red patches appear all over the body. In older children the skin folds are most often affected, especially the elbow creases and behind the knees. In adults the face and hands are more likely to be involved. The condition usually improves in childhood or sometime before the age of 25. Most people with atopic dermatitis have family members with similar problems. Serverity of the disease can be evaluated by an Eczema Area Severity Index (EASI) score (see, Hanifin, J M, et al., Exp. Dermatol., 10(1)11 - 18 (2001), the teachings of which are incorporated herein by reference for all purposes).

A condition similar to atopic dermatitis, but which affects mucosal tissues rather than the skin is asthma. Asthma is a chronic lung disease characterized by inflammation of the air passages. This condition is estimated to affect about 15 million Americans and can be severe and result in death if not treated. A number of factors can exacerbate asthma including, e.g., rapid changes in temperature or humidity, allergies, upper respiratory infections, exercise, stress or smoke (cigarette). Typical treatments include bronchodilators which are given orally or delivered as an aerosol (inhaled), and, for the most difficult cases, corticosteroids. Another example of a mucocutaneous inflammatory disorder is allergic rhinitis (hay fever). Allergic rhinitis is caused by a nasal inflammation in response to an irritant or an allergen. This condition can be seasonal or occur throughout the year (perennial). Typically, allergic rhinitis is treated by the administration of antihistamines either orally or locally (e.g., using nasal sprays).

Other examples of mucocutaneous inflammatory disorders include those that involve comification. Examples of such disorders include lamellar ichthyosis, acne, and rosacea.

Papulosquamous disorders are those characterized, as the name suggests, by scaly papules and plaques. Some of the more common papulosquamous disorders include psoriasis and lichen planus, both of which are manifested by a local inflammation of either the skin or a mucosal tissue (e.g., in the case of oral lichen planus).

Psoriasis is a persistent skin disease that got its name from the Greek word for “itch.” The skin becomes inflamed, producing red, thickened areas with silvery scales, most often on the scalp, elbows, knees, and lower back. Severe psoriasis may cover large areas of the body. Psoriasis is not contagious, and has some genetic basis as it is more likely to occur in people whose family members have it. In the United States about 2% of adults have psoriasis (four to five million people). Approximately 150,000 new cases occur each year. The cause of psoriasis is unknown. However, recent discoveries point to an abnormality in the functioning of key white cells in the blood stream triggering inflammation in the skin. Psoriasis is thus thought to be due, at least in part, to an abnormal immune reaction against some component of the skin. This leads to the local infiltration of inflammatory cells, including leukocytes, into the tissues, to the expression of cell adhesion molecules and to the up-regulation of inflammatory cytokines and growth factors. As a result, the two hallmark features of psoriasis are local inflammation and epidermal hyperproliferation. The combination of hyperproliferation with incomplete terminal differentiation leads to the formation of a thickened stratum corneum or plaques.

Psoriasis comes in many forms. Each differs in how severe it is, how long it lasts, where it is, and in the shape and pattern of the scales. The most common form begins with little red bumps. Gradually these bumps grow larger and scales form. While the top scales flake off easily and often, scales below the surface stick together. When they are removed, the tender, exposed skin bleeds. These small red areas then grow, sometimes becoming quite large.

In addition to psoriasis, other hyperproliferative skin disorders include, but are not limited to, basal cell carcinoma, squamous cell carcinoma (Bowen's disease), keratosis, such as actinic or seborrheic keratosis, and disorders of keratinization, such as ichthyosis and keratoderma. These hyperproliferative skin disorders result from the loss of the regulatory mechanisms that control the proliferation and differentiation of skin cells. Basal and squamous cell carcinomas are the most common forms of skin cancer. About 1.3 million cases of skin carcinomas are found in the United States per year. Both basal and squamous cell carcinoma affect the most external layer of the skin, the epidermis, and begin at the basal cell layer and at the upper cell layer of the epidermis, respectively. Although these skin carcinomas are slow growing and usually benign, they can, if not treated, grow and invade other tissues. In the year 2000, skin carcinomas will cause about 1,900 deaths in the United States.

Skin inflammation and irritation can also be caused by, for example, transdermal drug delivery, irritating drug delivery enhancers or irritating drug substances that are found in pharmaceutical products as well as in skin care products. Examples of irritating drug substances include, but are not limited to, retinoic acid and its derivatives and analogs, alpha-hydroxy acids and anthralin. The discomfort associated with the inflammation and/or irritation may affect the patient's compliance with the treatment and comfort during drug delivery.

In addition to changes resulting from inflammatory and hyperproliferative disorders, the appearance and characteristics of the skin also change as the body ages. Chronologically aged (intrinsically aged) mucocutaneous surfaces show a slight atrophy of the epidermis with straightening of the rete pegs thus weakening the dermal/epidermal junction measured by a decrease in the threshold for suction bullae. There is a moderate decrease in the number of Langerhans cells. Dryness of the skin is a common phenomenon. In the dermis there is a decrease in cell numbers and a decrease in elastic fibers and thus in skin elasticity. Capillaries are also fragile as evidenced by bruisability. Collagen metabolism is slower, and there is a progressive lowering in concentration of glycosaminoglycans. Sagging of the skin occurs. There is a decreased ability to mount inflammatory responses and an increase in the time of healing after injury.

Aging is accelerated in those areas exposed to environmental insults, such as, e.g., irritating substances and sunlight (ultraviolet radiation), due to the development of local skin inflammation. The skin aging process resulting from exposure to sunlight is known as “photoaging.” Photoaging accounts for about 80% of the visible changes of skin aging. It induces deep wrinkles not erased by stretching, pigmentary alterations with areas of hyper- and hypo-pigmentation (actinic lentigines and leukodermas), and a variety of benign, premalignant, and malignant neoplasms. The dermis shows evidence of chronic inflammation with increased cellularity and enlarged fibroblasts. Elastotic degeneration, known as the “grenz” zone, occurs in parts of the upper dermis. This zone is occupied by a basophilic fibrous material separating the dermis from the epidermis and is interpreted as a repair area. Glycosaminoglycan and elastin concentrations are increased.

There is a largely unmet medical need for effective and safe treatment of hyperproliferative, inflammatory, and related mucocutaneous disorders, including psoriasis, atopic dermatitis, basal and squamous cell carcinomas, asthma, allergic rhinitis and skin aging. Present treatments have often shown unfavorable side effects which limit efficacy (e.g., glucocorticoids), result in rebound of disease activity upon withdrawal of medication (e.g., glucocorticoids, cyclosporin A-like drugs), or increase the incidence of cancer (e.g., PUVA). Other drugs are inherently toxic (e.g., antimetabolites, such as methotrexate) and certain procedures are extremely inconvenient (e.g., coal tar treatments) or invasive (e.g., surgery). Present treatments for skin photodamage include, in particular, retinoids and alpha-hydroxy-acids which exhibit light sensitivity, limited efficacy, and untoward side effects. Excipients that are safe and contain active ingredients are rare and in high demand by the cosmetic and cosmeceutical industry.

In view of the foregoing, it is readily apparent that there is a great need in the art for new and effective treatments for a large number of inflammatory and hyperproliferative mucocutaneous disorders as well as for age-related skin disorders. The present invention addresses these and other needs.

### SUMMARY OF THE INVENTION

The present invention is based, in part, on the discovery that inhibitors of cholesterol biosynthesis and, in particular, inhibitors of HMG-CoA reductase, inhibitors of mevalonate metabolism, and inhibitors of protein prenylation are efficient for preventing and/or treating a variety of mucocutaneous disorders including, for example, hyperproliferative skin disorders and inflammatory skin disorders, such as atopic dermatitis, psoriasis, asthma and allergic rhinitis, as well as extrinsic skin aging and photoaging, skin photodamage and skin irritation. The present invention is further based on the surprising finding that inhibitors of HMG-CoA reductase and inhibitors of mevalonate metabolism can act synergistically.

As such, in one embodiment, the present invention provides a method for preventing and/or treating a skin disorder, wherein the skin disorder is selected from the group consisting of atopic dermatitis and skin photodamage, the method comprising administering to a patient in need thereof an HMG-CoA reductase inhibitor in a therapeutically effective amount. In one embodiment, exemplar HMG-CoA reductase inhibitors suitable for use in the method of the present invention include, but are not limited to, mevastatin, lovastatin, fluvastatin, pravastatin, simvastatin, dalvastatin, cerivastatin and atorvastatin. In another embodiment, suitable HMG-CoA reductase inhibitors useful in the method of the present invention include oxysterols and, in particular, 25-hydroxycholesterol. In yet another embodiment, the HMG-CoA reductase inhibitor is administered topically. In other embodiments, the HMG-CoA reductase inhibitor is formulated in a pharmaceutical composition.

The present invention also provides a method for preventing and/or treating a skin disorder, wherein the skin disorder is selected from the group consisting of atopic dermatitis, skin photodamage, extrinsic skin aging, skin photoaging and skin irritation, the method comprising administering to a patient in need thereof a protein prenylation inhibitor in a therapeutically effective amount. In one embodiment, peptide analogs are used to inhibit protein prenylation. Suitable peptide analogs include, but are not limited to, GGTI-286 and FTI-276. In another embodiment, suitable protein prenylation inhibitors include monoterpenes. Exemplar monoterpenes include, but are not limited to, d-limonene, perillic acid or perillyl alcohol. The protein prenylation inhibitor may be administered topically. In addition, the protein prenylation inhibitor may be formulated in a pharmaceutical composition.

The present invention further provides a method for preventing and/or treating skin inflammation, the method comprising administering to a patient in need thereof a protein prenylation inhibitor in a therapeutically effective amount. Again, suitable protein prenylation inhibitors include, but are not limited to, peptide analogs, such as GGTI-286 or FTI-276, and monoterpenes. Exemplar monoterpenes useful in the method of the present invention include, but are not limited to, d-limonene, perillic acid and perillyl alcohol. In a preferred embodiment, the protein prenylation inhibitor may be administered topically. The protein prenylation inhibitor may be formulated in a pharmaceutical composition.

The present invention is also directed to a pharmaceutical composition comprising a protein prenylation inhibitor and a topical carrier. Suitable protein prenylation inhibitors include, but are not limited to, peptide analogs, such as GGTI-286 and FTI-276, and monoterpenes, such as d-limonene, perillic acid and perillyl alcohol.

In another aspect, the present invention provides a method for preventing and/or treating skin disorders including, but not limited to, inflammatory skin disease, atopic dermatitis, skin photodamage, extrinsic skin aging, skin photoaging and skin irritation, the method comprising administering to a patient in need thereof a combination comprising at least two inhibitors of cholesterol biosynthesis in a therapeutically effective amount, wherein the first inhibitor is an HMG-CoA reductase inhibitor. In one embodiment, the second inhibitor is an inhibitor of mevalonate metabolism. The cholesterol biosynthesis inhibitors can be administered simultaneously or, alternatively, sequentially. The cholesterol biosynthesis inhibitors can further be administered topically. In one embodiment, suitable HMG-CoA reductase inhibitors include, but are not limited to, mevastatin, lovastatin, fluvastatin, pravastatin, simvastatin, dalvastatin, cerivastatin and atorvastatin. Other suitable HMG-CoA reductase inhibitors include oxysterols, such as 25-hydroxycholesterol. In some embodiments, the cholesterol biosynthesis inhibitors are formulated in a pharmaceutical composition.

The present invention also provides a method for preventing and/or treating a skin disorder, wherein the skin disorder is selected from the group consisting of hyperproliferative skin disorder and psoriasis, the method comprising administering to a patient in need thereof a combination comprising an HMG-CoA reductase inhibitor and an inhibitor of mevalonate metabolism. In one embodiment, the HMG-CoA reductase inhibitor and the mevalonate metabolism inhibitor are administered simultaneously. The HMG-CoA reductase inhibitor and the mevalonate metabolism inhibitor can be administered topically. As described above, HMG-CoA reductase inhibitors include, but are not limited to, statins, such as mevastatin, lovastatin, fluvastatin, pravastatin, simvastatin, dalvastatin, cerivastatin, atorvastatin, and oxysterols, such as 25-hydroxycholesterol. In some embodiments of the invention, the HMG-CoA reductase inhibitor and the mevalonate metabolism inhibitor are formulated in a pharmaceutical composition.

The present invention is further directed to a method for enhancing the potency of an anti-inflammatory drug, the method comprising administering to a patient in need thereof the anti-inflammatory drug and an excipient comprising an HMG-CoA reductase inhibitor. Again, HMG-CoA reductase inhibitors suitable for use in the method of the present invention include, but are not limited to, mevastatin, lovastatin, fluvastatin, pravastatin, simvastatin, dalvastatin, cerivastatin and atorvastatin. Additional HMG-CoA reductase inhibitors that are useful in the methods of the present invention include, but are not limited to, oxysterols and, in particular, 25-hydroxycholesterol. In another embodiment, the anti-inflammatory drug is selected from the group consisting of corticosteroids, salicylates, colchicine, para-aminophenol, propionic acid, piroxicam, ketorolac, ketoprofen, cyclooxygenase type II inhibitors and indomethacin.

In another aspect, the present invention provides a method for preventing and/or suppressing skin inflammation and irritation caused by transdermal or transmucosal drug delivery, irritating drug delivery enhancers or irritating drug substances, the method comprising administering to a patient in need thereof a therapeutic compound in combination with an excipient comprising an HMG-CoA reductase inhibitor. Suitable HMG-CoA reductase inhibitors include, but are not limited to, mevastatin, lovastatin, fluvastatin, pravastatin, simvastatin, dalvastatin, cerivastatin and atorvastatin. Other suitable HMG-CoA reductase inhibitors include, but are not limited to, oxysterols and, in particular, 25-hydroxycholesterol. In yet another embodiment, the therapeutic compound is selected from the group consisting of glycerol, corticosteroids and salicylates.

### BRIEF DESCRIPTION OF THE DRAWINGS

FIG. 1 illustrates the reduction of ear swelling in the mouse acute irritant model following administration of mevastatin and oxysterols.

FIG. 2 illustrates the inhibition of T cell proliferation in vitro, following administration of (A) mevastatin and oxysterols and (B) oxysterol combined with lanolin.

FIG. 3 shows that the inhibition of T cell proliferation by mevastatin is reversed by mevalonate.

FIG. 4 shows that the inhibition of T cell proliferation by (A) 25-hydroxycholesterol and (B) oxysterol combined with lanolin is not reversed by mevalonate.

FIG. 5 illustrates that the inhibition of T cell proliferation by mevastatin is potentiated by (A) 25-hydroxycholesterol and (B) oxysterol combined with lanolin.

FIG. 6 shows that the GGTI-286 geranylgeranyl transferase inhibitor effectively blocks T cell proliferation.

FIG. 7 illustrates the ability of mevastatin to reduce topical TPA challenge-induced acute inflammation.

FIG. 8 illustrates the ability of topical formulations containing (A) lovastatin, oxysterol or both; and (B) simvastatin to suppress ear swelling in the DNFB-induced contact hypersensitivity reactions in mice. (C) shows the ability of lovastatin to inhibit oxazolone-induced contact hypersensitivity reactions in mice.

### DETAILED DESCRIPTION OF THE INVENTION AND PREFERRED EMBODIMENTS

I. INTRODUCTION

This invention relates, in part, to the discovery that the administration of synthetic or naturally occurring inhibitors of cholesterol metabolism and, in particular, inhibitors of the mevalonate biosynthesis and/or metabolism, effectively prevent and/or treat skin irritation, hyperproliferative skin disorders and mucocutaneous inflammatory disorders and conditions including, e.g., atopic dermatitis, basal cell carcinoma, squamous cell carcinoma, asthma, allergic rhinitis and psoriasis. Moreover, inhibitors of cholesterol metabolism have now been discovered to prevent and reverse the signs of skin aging and, in particular, skin photoaging and skin photodamage. The compounds of the invention can thus be used as active cosmeceutical ingredients to treat and prevent skin photodamage and skin irritation. The present invention is further directed to the use of inhibitors of cholesterol metabolism to protect from and/or to treat skin irritation caused by transdermal and transmucosal drug delivery and as active excipients to enhance the potency of antiinflammatory products. The compounds of the invention can be formulated in various ways for optimal delivery and efficacy. In preferred embodiments, the cholesterol biosynthesis inhibitors of the present invention are inhibitors of the mevalonate biosynthesis and/or metabolism.

II. MUCOCUTANEOUS DISORDERS

The methods of the present invention can be used to prevent and/or treat inflammatory skin diseases (e.g., atopic dermatitis, eczema, contact dermatitis and allergic dermatitis), hyperproliferative skin diseases (e.g., psoriasis, basal cell carcinoma and squamous cell carcinoma), and skin irritation. Such conditions are well known to those of skill in the art and are described, e.g., in Champion et al., Eds. (1998) “Textbook ofDermatology”, Blackwell Science, or in information provided by any of a number of organizations such as the American Academy of Dermatology (see, e.g., http://www.dermfnd.org/) and the American Cancer Society (see, e.g., http://www.cancer.org/). Further, the compounds and compositions of the present invention can be used to treat any symptom associated with any of these diseases or conditions, such as inflammation, redness, itching, pimples, crusts, scabs, dryness, burning, oozing, fluid, e.g., pus, discharge, pustules, blistering, rashes, disfiguration, scaling, dandruff, papules, plaques, lesions, thickenings, shedding, bumps, flaking, bleeding, tenderness, cuts, scratches, irritation, swelling, blebs, vesicles, elevations, scarring, wrinkling, freckling, yellowing, blood vessel dilation and others.

The compounds and compositions of the present invention are also useful for preventing and/or treating mucocutaneous inflammatory diseases such as asthma and allergic rhinitis as well as their associated symptoms. Descriptions of such conditions can be found in the Asthma and Allergy Foundation of America (see, e.g., http://www.aafa.org/) and are well known to those of skill in the art. Asthma is characterized by paradoxical narrowing of the bronchi that results in breathing difficulties. Typical symptoms associated with asthma include, e.g., wheezing, breathing difficulties, tightness of the chest, dry cough and shortness of breath after exercise. The compounds of the present invention can also be used to treat allergic rhinitis (hay fever). Allergic rhinitis results from an inflammatory reaction that occurs in the nasal passages in response to an allergic stimulus. Symptoms associated with allergic rhinitis include, e.g., sneezing, nasal congestion, nasal itching, nasal discharge and itching of the roof of the mouth and/or ears.

The compounds and compositions of the present invention can also be used to prevent and/or treat skin aging, in particular extrinsic skin aging, as well as any symptoms associated with skin aging. Such symptoms include, for example, appearance of wrinkles and/or fine lines, slackening of cutaneous and subcutaneous tissue, sagging of the skin, atrophy of the epidermis, increased dryness of the skin, decrease in skin elasticity, increased fragility of capillaries, increased time of healing after injury, pigmentary alterations with areas of hyper-and hypopigmentation, appearance of a variety of benign, premalignant, and malignant neoplasms, etc. Furthermore, at the histological level, aging results in thinning and deterioration of the skin, as well as in the reduction in cells and in blood supply, and a flattening in the junction between the dermis and epidermis.

In addition, the compounds and compositions of the present invention can be used to prevent and/or treat skin photodamage and any associated symptoms. Skin photodamage occurs with aging due to prolonged or repeated exposure to ultraviolet radiation. Signs of skin photodamage include, for example, wrinkling, yellowing, appearance of spots and mottling, elastosis, appearance of lines, leathery or dry appearance of the skin, and premature aging of the skin. At the histological level, skin photodamage may be reflected in tangled, thickened, abnormal elastic fibers, decreased collagen and increased glycosaminoglycan content (Tanaka et al. (1993) Arch. Dermatol. Res. 285:352-355).

The inhibitors of cholesterol biosynthesis of the present invention are efficient for preventing and/or treating mucocutaneous inflammation and irritation caused, for example, by transdermal or transmucosal drug delivery, irritating drug delivery enhancers or irritating drug substances.

The compounds and compositions of the present invention can also be used as excipients to enhance the potency of antiinflammatory drugs, such as corticosteroids, salicylates, colchicine, para-aminophenol, propionic acid, piroxicam, ketorolac, ketoprofen, cyclooxygenase inhibitors, indomethacin, etc.

III. INHIBITORS OF CHOLESTEROL METABOLISM

A. Inhibitors

Cholesterol is synthesized from acetic acid via a relatively large number of reaction steps. Major steps in cholesterol biosynthesis include the synthesis of mevalonate by HMG-CoA reductase, which is the rate limiting step in the generation of cholesterol and other essential isoprenoid products, including dolichol, ubiquinone, and isoprenylated proteins. Downstream of mevalonate, steps in cholesterol biosynthesis include the synthesis of isopentenyl pyrophosphate, the synthesis of famesyl pyrophosphate, the synthesis of squalene, and finally the synthesis of cholesterol. Cholesterol synthesis can thus be modulated by modulating the various steps in the cholesterol biosynthesis pathway.

As described above, the present invention is directed to methods for preventing and/or treating a variety of hyperproliferative and inflammatory mucocutaneous disorders, as well as disorders associated with skin aging and skin irritation. Compounds for use with the methods of the present invention include, but are not limited to, inhibitors of cholesterol biosynthesis acting at any of the steps of the biosynthesis pathway, and in particular inhibitors of HMG-CoA reductase and inhibitors of protein prenylation, which are especially useful for treating hyperproliferative conditions such as ichthyosis, inhibitors of famesyl transferase. Such compounds can be used individually or in combination with another (or more) cholesterol biosynthesis inhibitor(s). Useful compounds include those that downregulate the expression of a gene encoding the target protein as well as those that inhibit the function of the target protein itself. The compounds of the present invention can further be used as excipients to enhance the effect of anti-inflammatory drugs or of compounds that are used to treat skin irritation. The cholesterol biosynthesis inhibitors of the present invention can be naturally occurring or synthetic.

1. Inhibitors of HMG-CoA Reductase

Mevalonate synthesis is a reversible step catalyzed by the 3-hydroxy-3- methylglutaryl coenzyme A reductase enzyme (referred to herein as HMG-CoA reductase). It has now been discovered that inhibition of HMG-CoA reductase is useful for treating a variety of hyperproliferative and inflammatory mucocutaneous disorders.

The HMG-CoA reductase inhibitors of the invention include any compound that exhibits HMG-CoA reductase inhibitory activity or compounds that interfere with the HMG-CoA reductase gene expression. Oxysterols are one example of HMG-CoA reductase inhibitors that act by inhibiting HMG-CoA reductase gene expression and that can be used in the methods of the present invention. Oxysterols are described below, due to their broader effects on cholesterol biosynthesis. In addition to oxysterols, preferred HMG-CoA reductase inhibitors include, but are not limited to, 3,5-dihydroxy carboxylic acids of the statin type, such as mevastatin (also referred to as compactin or ML-236B), lovastatin (also referred to as MK-803 or Mevinolin), simvastatin (also referred to as MK-733 or Synvinolin), fluvastatin, pravastatin, dalvastatin (also referred to as RG 12561), cerivastatin and atorvastatin and their δ-lactones, as well as derivatives and salts thereof.

2. Inhibitors of Mevalonate Metabolism

In addition to HMG-CoA reductase, other enzymes in the mevalonate metabolism can be targeted for inhibition and include, e.g., HMG-CoA synthase, and enzymes in the downstream mevalonate metabolism, such as farnesyl pyrophosphate synthase and enzymes catalyzing protein prenylation (e.g., famesyl pyrophosphate transferase and geranylgeranyl pyrophosphate transferase type I and II). As used herein, “mevalonate metabolism” refers to any of the steps in the cholesterol biosynthesis pathway that is located between the synthesis of mevalonate and the synthesis of farnesyl pyrophosphate.

Some β-lactones and β-lactams can be used to inhibit HMG-CoA synthase (see, e.g., U.S. Pat. Nos. 4,983,597 and 4,751,237; and EP 0462667).

Enzymes catalyzing steps of the dowstream mevalonate metabolism include, for example, mevalonate kinase, phosphomevalonate kinase and diphosphomevalonate decarboxylase, which catalyzes the last step in the conversion of mevalonate into isopentenyl pyrophosphate. Inhibitors of any of these steps can be used in the methods and compositions of the present invention.

Squalene is synthesized from isopentenyl pyrophosphate in a series of reactions that involve enzymes such as prenyl transferase, geranylgeranyl pyrophosphate transferase and farnesyl pyrophosphate transferase. Prenyl transferases transfer farnesyl pyrophosphate and geranylgeranyl pyrophosphate to proteins (e.g., small GTP binding proteins, such as, e.g., members of the rho and ras family). Specific inhibitors of geranylgeranyl pyrophosphate transferase and farnesyl pyrophosphate transferase for use in the methods and compositions of the present invention include, but are not restricted to, various peptide analogues, (e.g., GGTI-286 and FTI-276) and synthetic compounds. Monoterpenes, including, e.g., d-limonene, perillic acid, and perillyl alcohol, have also been shown to inhibit the prenylation of 21-26 kDa proteins and inhibit DNA synthesis in lymphocytes and, thus, can also be used in the methods and compositions of the present inventions.

3. Other Inhibitors of Cholesterol Biosynthesis

Further inhibitors of cholesterol biosynthesis that can be used in the methods of the present invention include, for example, oxysterols (e.g., 25-hydroxycholesterol) that inhibit the sterol-sensitive proteolytic cleavage and activation of SREBP, a transcription factor which regulates a number of genes encoding proteins involved in cholesterol homeostasis, i.e., HMG-CoA synthase, HMG-CoA reductase, famesyl diphosphate synthase, squalene synthase, and the LDL receptor.

Other suitable inhibitors, useful in the methods of the present invention, include those that modulate the steps of cholesterol biosynthesis downstream of the synthesis of squalene. Such inhibitors include, but are not limited to, inhibitors of the enzyme squalene synthetase (e.g., isoprenoid-(phosphinylmethyl)-phosphanates; see, e.g., EP 0409181; and Biller et al. (1991) J Med. Chem. 34:1912), of the enzyme squalene epoxidase (e.g., allylamines, such as naftifine and terbinafine; see, e.g., Horie et al. (1990) J Biol. Chem. 265:18075-18078) and of the enzyme 2,3-epoxysqualene-lanosterol cyclase. Inhibitors of the 2,3-epoxysqualene-lanosterol cyclase include, e.g., aminoalkoxybenzene derivatives (see, EP 0410359), piperidine derivatives (see, (1992) J Org. Chem. 57:2794-2803), decalins, azadecalins and indane derivatives (see, WO 89/08450; (1981) J Biol. Chem. 254:11258-11263; (1988) Biochem. Pharmacology 37:1955-1964; and JP 64/003144), 2-aza-2,3-dihydrosqualene and 2,3-epiminodsqualene ((1985) Biochem. Pharmacology 34:2765-2777), squalenoid epoxide vinyl ethers ((1988) J. Chem. Soc. Perkin Trans. I:461), 29-methylidene-2,3-oxidosqualene ((1991) J Amer. Chem. Soc. 113:9673-9674), and N,N-disubstituted arylcycloalkylamines (U.S. Pat. No. 5,455,273). Inhibitors of the enzyme lanosterol-14α-demethylase can further be used in the context of the present invention, including, but not limited to, antimycotics of the azole type, such as N-substituted imidazoles and triazoles (e.g., ketoconazole and fluconazole).

The cholesterol biosynthesis inhibitors, in particular the inhibitors of mevalonate biosynthesis and/or metabolism, can be used alone, or in conjunction with other topical therapeutic agents known to be efficient for treating any of the above-listed mucocutaneous disorders. Furthermore, two or more inhibitors of cholesterol biosynthesis can be used in combination to treat any of the mucocutaneous disorders of the present invention.

4. Other Pathways Affected by the Compounds of the Invention

Some of the inhibitors described herein also affect other metabolic or biochemical pathways. The present invention thus also relates to the discovery that the effects of the above-listed inhibitors on hyperproliferative and inflammatory mucocutaneous disorders, as well as on skin irritation and skin aging may be due, at least in some cases, to the modulation of pathways other than the cholesterol biosynthesis pathway.

For example, lovastatin is an inhibitor of the HMG-CoA reductase enzyme and is used clinically for the treatment of hypercholesterolemia. Lovastatin has been shown to also affect several signal transduction pathways and immune cell functions in vitro by suppressing the synthesis of non-sterol isoprenoid intermediates. In particular, the present invention relates to the discovery that lovastatin inhibits the expression of IL-2 in the presence of an excess of mevalonate. Lovastatin can thus affect lymphocytes through a novel mechanism, independent of its role as an inhibitor of HMG-CoA reductase and isoprenoid synthesis.

B. Making the Inhibitors

The inhibitors of cholesterol biosynthesis, in particular the inhibitors of mevalonate biosynthesis and/or metabolism, for use with the methods of the present invention are either naturally occurring or synthetic.

Various HMG-CoA reductase inhibitors are naturally occurring as fungal products. For example, mevastatin is produced by Monascus purpureus Went yeast fermented on rice. Fungal extracts or extracts of commercially available products, e.g., Chinese fermented red rice, may also serve as sources of HMG-CoA reductase inhibitors. HMG-CoA reductase inhibitors can further be isolated from microfungus of the genus Aspergillus (see, e.g., U.S. Pat. Nos. 4,231,938; 4,294,296; and 4,450,171).

Protein prenylation inhibitors can also come from natural sources such as fungi and plant terpenes. Furthermore, oxysterols are present in lanolin and lanolin oil, and the corresponding oxyphytosterols and oxyergosterols can be obtained from plant and fungal sources, respectively.

Alternatively, these substances can be chemically synthesized using techniques well known to those of skill in the art (see, e.g., U.S. Pat. No. 5,455,273 and 5,075,327). Oxysterols can also be produced by oxidation of appropriate starting materials, e.g., cholesterol, lanosterol, stigmasterol, sitosterol, ergosterol.

C. Synergy

The compounds of the present invention can be used to prevent and/or treat a variety of skin disorders either individually or in combination.

Surprisingly, the inhibitors of HMG-CoA reductase (e.g., mevastatin) and the inhibitors of mevalonate metabolism (e.g., oxysterols, protein prenylation inhibitors) of the present invention achieve synergistic results, i.e., are synergistic.

In the context of the present invention two active compounds are “synergistic” when the effectiveness of the two components in a mixture is more than additive, i.e., the effectiveness is greater than the equivalent concentration of either component alone. For example, the effectiveness of the combination therapy of an HMG-CoA reductase inhibitor and an inhibitor of mevalonate metabolism is synergistic.

The HMG-CoA reductase inhibitors and the inhibitors of mevalonate metabolism of the present invention can thus be combined to produce biological effects greater than the sum of the individual agents alone. One advantage of this synergistic effect is that the dosage of each inhibitor can be reduced to achieve the desired therapeutic effect and thus the side effects of each compound can be concomitantly reduced.

D. Measuring the Effect of the Cholesterol Inhibitors

The effectiveness of the cholesterol biosynthesis inhibitors of the present invention can be tested in vitro or in vivo using standard methods well known to those of skill in the art. The ability to inhibit an enzyme of interest can also be measured using standard assays known to and used by those of skill in the art.

The effects on the immune response of a compound of interest or of a combination of compounds can be tested in vivo, for example, by evaluating murine thymic T cells proliferation and IL-2 production or gene expression. Methods to measure T cell proliferation and IL-2 production are standard and well known to those of skill in the art. Exemplar animal models for estimating the effects of the compounds of the present invention include, but are not limited to, the mouse acute irritant model, the allergic contact hypersensitivity model, etc. Other suitable models include, for example, the inbred strain of NC/Nga mice which, when reared under non-pathogen-free conditions, develops chronic relapsing skin inflammation. Injection of Balb/c mice with Shistosoma japonica glutathione-S-transferase leads to the development in the injected mice of a systemic dermatitis, providing a useful model of allergic dermatitis. Additional models for skin allergies can be obtained by regularly applying to the ears of mice 2,4-dinitrofluorobenzene, which induces an allergic cutaneous response in the mice (Nagai et al. J Pharmacol. Exp. Therapeutics 288:43-50). Other suitable models for testing the effects of the compounds of the present invention on atopic dermatitis include, but are not limited to, the repetitive epicutaneous sensitization of mice with the antigen ovalbumin. Examples of such screening models are disclosed herein.

V. PHARMACEUTICAL COMPOSITIONS AND ADMINISTRATION

The present invention also provides pharmaceutical compositions for the administration of the herein-described cholesterol inhibitors to a patient in need thereof. In the context of the present invention, the term “patient” refers to an organism to which the compounds of the invention can be administered. Preferably, a patient is a mammal, e.g., a rodent, a primate or a human. A patient may be afflicted with a disease, or may be free of detectable disease in which case the compounds and compositions of the present invention are administered prophylactically. The compositions of the present invention can be administered to patients with a hyperproliferative or an inflammatory mucocutaneous disorder or condition, or suffering from skin irritation or from one or more symptoms associated with skin aging and/or skin photodamage.

The cholesterol inhibitors of the present invention, in particular the inhibitors of mevalonate biosynthesis and/or metabolism, can be formulated to be administered using any of a variety of routes, including, e.g., intravenous, intramuscular, transmucosal, oral or topical administration, such as, e.g., subcutaneously or transdermally, for prophylactic and/or therapeutic treatment.

The present compounds can be incorporated into a variety of compositions for therapeutic and/or prophylactic administration. A number of suitable formulations for use in the present invention are found in Remington's Pharmaceutical Sciences (Mack Publishing Company, Philadelphia, Pa., 17th ed. (1985) and in Dermatological Formulations: Percutaneous absorption, Barry (Ed.), Marcel Dekker Inc. (1983), both incorporated herein by reference. Moreover, for a brief review of methods for drug delivery, see, Langer, Science 249:1527-1533 (1990), which is also incorporated herein by reference. The pharmaceutical compositions described herein can be manufactured in a manner that is known to those of skill in the art, i.e., by means of conventional mixing, dissolving, levigating, emulsifying, encapsulating, entrapping or lyophilizing processes. It will be appreciated that the present methods and excipients are merely exemplary and are in no way limiting.

More particularly, these compounds can be formulated into preparations in solid, semi-solid, or liquid forms suitable for local or topical administration, such as gels, water soluble (e.g., K-Y) jellies, creams, lotions, suspensions, foams, powders, slurries, ointments, solutions, oils, pastes, sprays, emulsions, saline solutions, dimethylsulfoxide (DMSO)-based solutions, inhalers and aerosols. In general, carriers with higher densities, such as K-Y jelly, are capable of providing an area with a prolonged exposure to the active ingredients. In contrast, a solution formulation provides more immediate exposure of the active ingredient to the chosen area, although the effects generally do not last as long.

In addition to the formulations described supra, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients, which are compounds that allow increased penetration of, or assist in the delivery of, therapeutic molecules across the stratum comeum permeability barrier of the skin. There are many of these penetration enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, ORGELASE, water, calcium carbonate, calcium phosphate, various sugars, starches, cellulose derivatives, gelatin, and polymers such as polyethylene glycols.

For enteral administration the compounds of the invention can be administered in either single or multiple dosages. The compounds of the invention may be administered in combination with pharmaceutically acceptable carriers in a variety of dosage forms. For example, capsules, lozenges, hard candies, powders, sprays, aqueous suspension, elixirs, syrups, and the like may be formulated with various pharmaceutically acceptable inert carriers. Such carriers include solid diluents or fillers, sterile aqueous media and various non-toxic organic solvents. In general, the compounds of the invention will be included in oral dosage forms at concentration levels ranging from about 0.5% to about 90% by weight of the total composition, in amounts which are sufficient to provide the desired unit dosage.

Tablets may contain various excipients such as sodium citrate, calcium carbonate and calcium phosphate, along with various disintegrants such as starch (preferably potato or tapioca starch), alginic acid and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in soft and hard-filled gelatin capsules; preferred materials in this connection would also include lactose or milk sugar as well as high molecular weight polyethylene glycols. When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

For parenteral use, the compounds of the invention may be formulated by means known in the art using suitable dispersing or wetting agents and suspending agents. A sterile injectable formulation can also be a solution or suspension in a non-toxic parenterally acceptable diluent or solvent, for example, as a solution in 1,3-butandiol. Among the acceptable vehicles and solvents are water, Ringer's solution and isotonic NaCl solution, fixed oils (including synthetic mono-or di-glycerides), fatty acids (such as oleic acids), and mixtures thereof.

Pharmaceutical compositions suitable for use in the present invention include compositions wherein the active ingredients are contained in a therapeutically or prophylactically effective amount. The amount of compound or composition administered will, of course, be dependent on the subject being treated, on the subject's weight, the severity of the affliction, the manner of administration and the judgment of the prescribing physician. Determination of an effective amount is well within the capability of those skilled in the art, especially in light of the disclosure provided supra.

As used herein, “effective amount,” or “therapeutically effective amount” refers to an amount of any of the present compounds that results in treatment of the medical condition, i.e., reduction in pain, redness, inflammation, or any other symptom. Reduction in pain is subjectively determined by the user, and will include any perceptive lessening of pain. Alternatively, an “effective amount” may be determined by monitoring reduction in any detectable symptom of the condition, such as the degree of swelling, inflammation, redness, size of the affected area, etc. In the context of the present invention, “prophylactically effective amount” refers to an amount of any of the present compounds that prevents the development or relapse of a medical condition. For example, a “prophylactically effective amount” is an amount that protects a subject from the deleterious effects of ultraviolet irradiation and that thus prevents photodamage and/or the appearance of signs of skin aging. In diseases that relapse periodically, such as atopic dermatitis or psoriasis, administration of a prophylactically effective amount of a compound of the invention may be an amount useful for preventing the relapse of the condition.

For any compound used in the method of the invention, a therapeutically effective dose can be estimated initially from animal models (described supra), well-known to those of skill in the art. Such information can be used to more accurately determine useful doses in humans. Initial dosages can also be estimated from in vitro or in vivo data.

Initial dosages can also be formulated by comparing the effectiveness of the compounds described herein in model assays with the effectiveness of known drugs. For instance, initial dosages can be formulated by comparing the effectiveness of the compounds described herein in model assays with the effectiveness of other compounds that have shown efficacy in treating the present conditions. In this method, an initial dosage can be obtained by multiplying the ratio of effective concentrations obtained in the model assay for the present compound and the control compound by the effective dosage of the control compound. For example, if the present compounds are twice as effective in a model assay as a known compound (i.e., the EC50 of the compound is equal to one-half the EC50 of the known compound in the same assay), an initial effective dosage of the compound of the present invention would be one-half the known dosage for the known compound. Using these initial guidelines one having ordinary skill in the art could readily determine an effective dosage in humans or other mammals.

Moreover, toxicity and therapeutic efficacy of the compounds described herein can be determined by standard pharmaceutical procedures in cell cultures or experimental animals, e.g., by determining the LD50 (the dose lethal to 50% of the population) and the ED50 (the dose therapeutically effective in 50% of the population). The dose ratio between toxic and therapeutic effect is the therapeutic index and can be expressed as the ratio between LD50 and ED50. Compounds which exhibit high therapeutic indices are preferred. The data obtained from these cell culture assays and animal studies can be used in formulating a dosage range that is appropriate for use in humans. The dosage of such compounds lies preferably within a range of concentrations that include the ED50 with little or no toxicity. The dosage may vary within this range depending upon the dosage form employed and the route of administration utilized. The exact formulation, route of administration and dosage can be chosen by the individual physician in view of the patient's condition (see, for example, Fingl et al. “The Pharmacological Basis of Therapeutics” Ch. 1, p. 1 (1975)).

Dosage amount and interval may be adjusted individually to provide levels of the active compound which are sufficient to maintain therapeutic effect. One having skill in the art will be able to optimize therapeutically effective local dosages without undue experimentation.

The preferred concentration of the compounds included in the herein-provided combinations, or any supplementary active agent, in the herein-described pharmaceutical compositions ranges from about 0.01% to about 60.0% with from about 0.025% to about 30.0% being the most preferred. In terms of weight of the compound per volume of a carrier, the present compounds, or supplementary agents, can be present at a concentration ranging from about 0.00001 to about 100 mg compound/1 ml of a carrier. In a preferred embodiment, the compound is present in an amount from about 0.0001 mg to about 10 mg/1 ml of a carrier. In yet another embodiment, the compound is present in an amount from about 0.001 to about 5 mg/1 ml of carrier.

In pharmaceutical dosage forms, the compounds may be administered in the form of their pharmaceutically acceptable salts, or they may also be used alone or in appropriate association, as well as in combination with other pharmaceutically active compounds. Agents of particular use in the formulations of the present invention include, for example, local anesthetics, counterirritants, anti-inflammatory agents, or any agent that has a therapeutic effect for hyperproliferative skin diseases or inflammatory mucocutaneous diseases or conditions.

The preferred anti-inflammatory agents include, but are not limited to, prescription and nonprescription topical and aerosol corticosteroids, non-steroidal anti-inflammatory agents including salicylates, colchicine, para-aminophenols, propionic acids and macrolide immunosuppressives with dapsone, clobetasol, halobetasol, diflorasone, piroxicam, ketorolac, ketoprofen, indomethacin and specific cyclooxygenase inhibitors.

The preferred counterirritants include, but are not limited to, glycerol, corticosteroids and salicylates. The preferred anesthetics include, but are not limited to, amide caines and counterirritants with lidocaine, cocaine, bupivicaine, mepivicaine, etidocaine, chloroprocaine, proparacaine, tetracaine, benzacaine, prilocaine, benoxinate, dibucaine, dyclonine, pramoxine, menthol, resorcinol, thymol and camphor.

Any other compound that has potential efficacy in the treatment of the present conditions can also be used.

The present formulations can be administered to treat an existing disease or condition, or can be used prophylactically. In prophylactic applications, compositions containing the present compounds can be administered to a patient that is not already in a disease state in order to enhance the patient's resistance or to retard the progression of disease or condition. Such an amount is defined as a “prophylactically effective dose or amount.” In this use, the precise amounts again depend upon the patient's state of health and general level of immunity, but are generally present at a concentration ranging from about 0.00001 to about 100 mg compound/1 ml of a carrier. In a preferred embodiment, the compound is present in an amount from about 0.0001 mg to about 10 mg/1 ml of a carrier. In yet another embodiment, the compound is present in an amount from about 0.001 to about 5 mg/1 ml of carrier.

The present compositions can be administered to a patient using a variety of routes, such as oral, parenteral or local routes. The present compositions are typically administered to a patient as a local application, where “local application,” or “locally applied,” refers to the administration of a composition at the local site of the disease, whether by local injection, topical administration, or any such method that results in a relatively high concentration of the present compounds at the site of the disease. As such, administration of the compounds can be achieved in various ways, including by topical application of the composition to the site of the disease or condition, i.e., direct application of a formulation to the affected skin or mucous membrane. In addition, compositions can be formulated for injection and injected locally at the site of the disease or condition, e.g., local subcutaneous injection at the site of the disease.

The present compositions can be applied to any site of any of the present conditions, including localized conditions or conditions affecting large areas of the body or even covering the entire body, can be applied to the skin and/or to mucous membranes, and can be applied to any affected part of the body, including the face, forehead, chin, eyes, eyelids, eyebrows, nose, skin near the nose, cheeks, ears, mouth, tongue, inside of the cheeks, gums, head, hair, scalp, neck, chest, back, lower back, armpit, skin folds of armpit, elbow, elbow fold, wrists, ankles, legs, arms, insides of wrists, insides of arms, nails, knees, area behind knees, hands, feet, palms, soles, fingers, toes, genitals, or any other affected part of the body.

The present compositions can be administered one time or multiple times, depending on the compound, the severity of the condition, and the initial response of the condition to the treatment. For example, the compositions can be administered 1, 2, 4, or more times per day, and can be administered every 1, 2, 4, 7, or more days. Such treatments can be administered for a limited duration, or indefinitely until the condition has resolved. The compositions can be applied locally as a “leave on” product, meaning that the composition is applied to the patient and allowed to remain indefinitely at the site of application, or as a “wash off” product, meaning that the composition is allowed to remain at the site of application for a limited amount of time, e.g., for a certain number of seconds, minutes, hours, etc.

It will be appreciated that the present methods of treatment can be applied alone or in combination with other surgical or non-surgical treatments for these conditions.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

### Example 1

### Inhibition of 12-O-Tetradecanoyl-13-Phorbol Acetate (TPA)-Induced Ear Swelling by Mevastatin

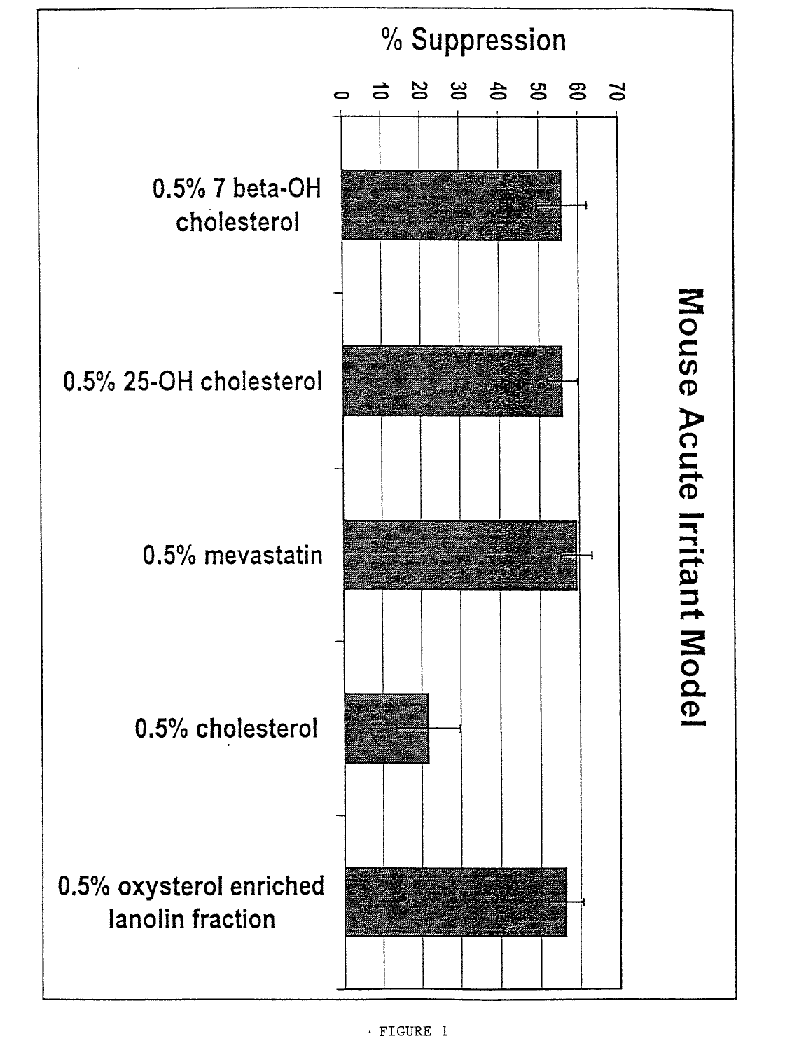

Mevastatin was tested topically at 0.5% with several oxysterols known to downregulate HMG-CoA reductase expression, including 25-hydroxycholesterol, in the mouse acute irritant dermatitis model. TPA was applied topically to the ears of mice and the resultant swelling response was measured 24 hr later. Compounds or vehicle alone were topically applied to ears 1 and 6 hr following TPA treatment. The data was expressed as a percent inhibition of the swelling response in the presence of compounds as compared to ears treated with vehicle only. This experiment showed that mevastatin significantly inhibited TPA-induced ear swelling (FIG. 1).

### Example 2

### Inhibition of ConA-Induced Murine Thymocyte Proliferation by Mevastatin

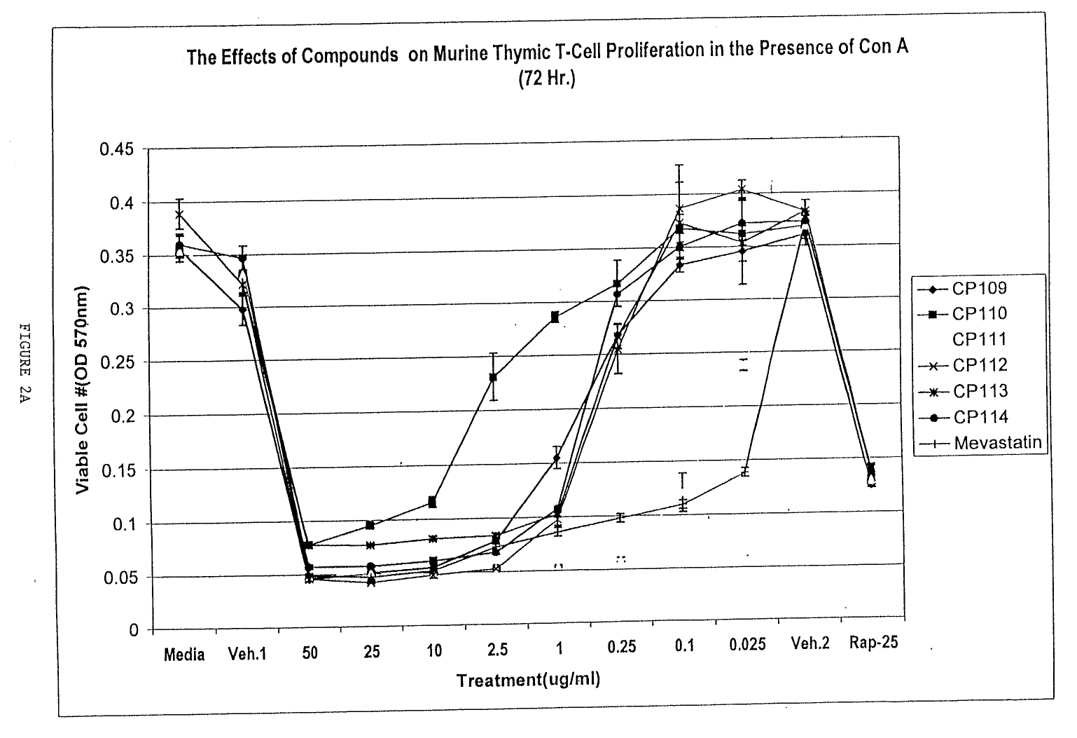

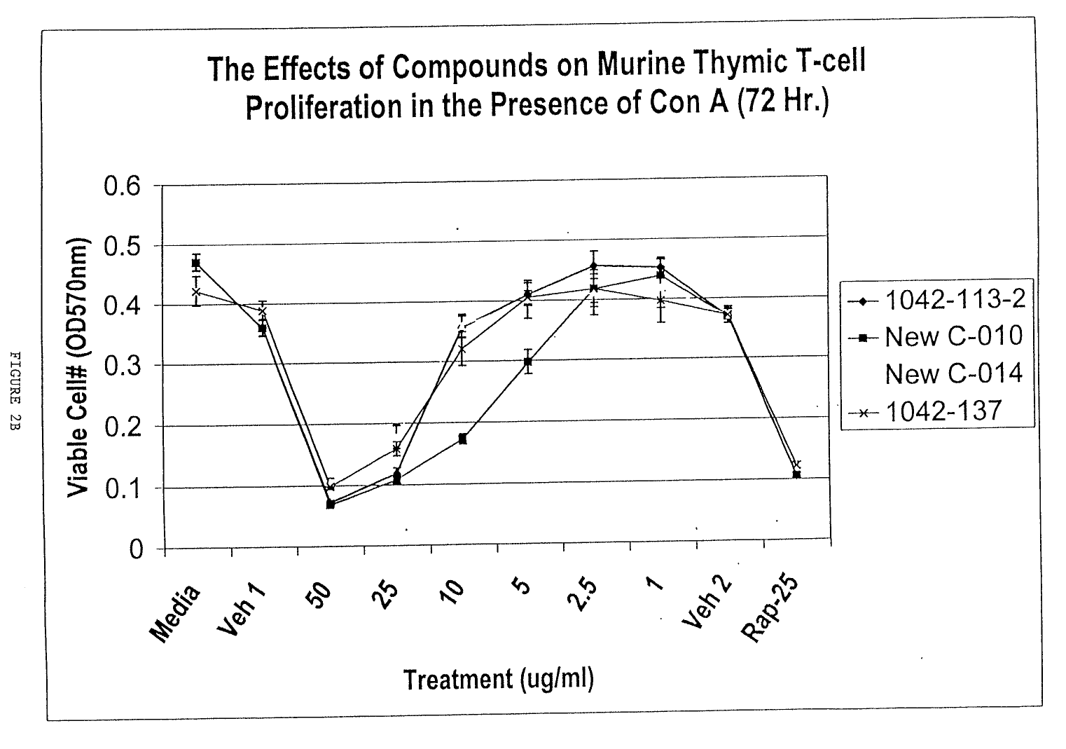

Murine thymic T cells were stimulated with Concanavalin A and incubated for 72 hr, and proliferation was then assessed by measuring viable cell number using the MTT assay (OD 570 nm proportional to viable cell number). Different concentrations of mevastatin and oxysterol alone (CP compounds; Panel A) or oxysterol combined with lanolin (Panel B) were added to the cells 1 hr following ConA treatment (FIG. 2). This experiment showed that mevastatin was able to potently inhibit murine thymocyte proliferation induced by the mitogen ConA.

### Example 3

### Inhibition of T Cell Proliferation by Mevastatin is Reversed by Mevalonate

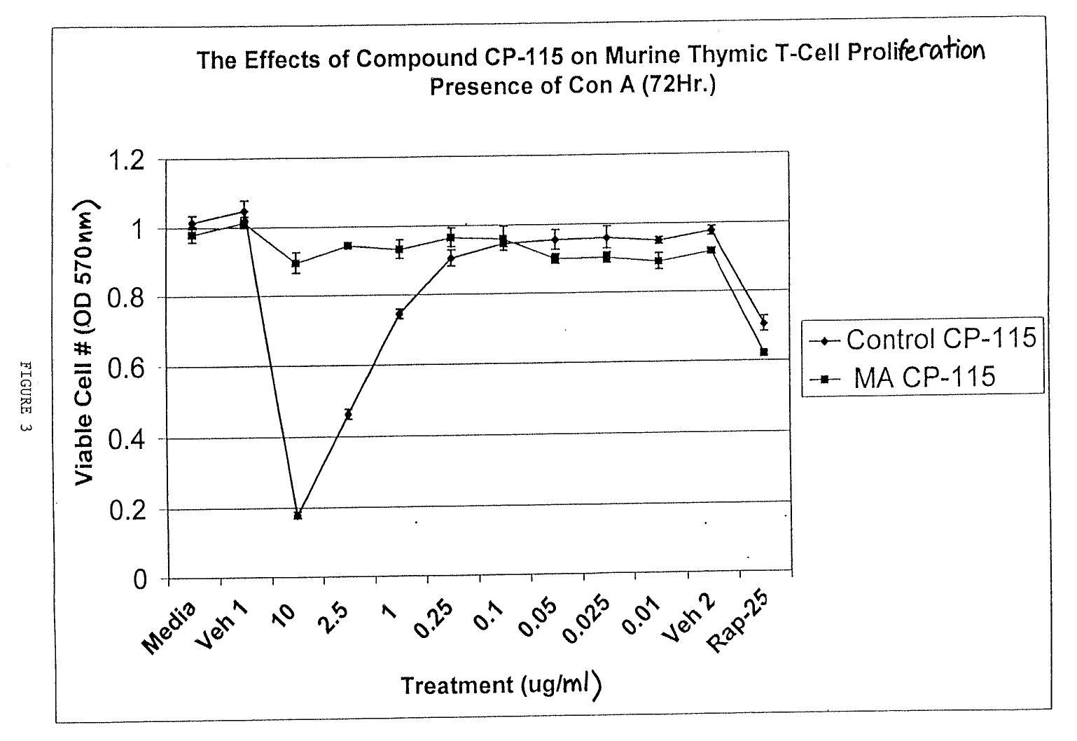

T cells were incubated with different concentrations of mevastatin (CP 115) alone or in the presence of 1 mM mevalonate (MA). The inhibition of T cell proliferation by mevastatin was prevented by addition of mevalonic acid, the product of the HMG-CoA reductase reaction (FIG. 3). The inhibition of T cell proliferation by mevastatin was thus reversed by mevalonate.

### Example 4

### Inhibition of T Cell Proliferation by Oxysterols is Not Reversed by Mevalonate

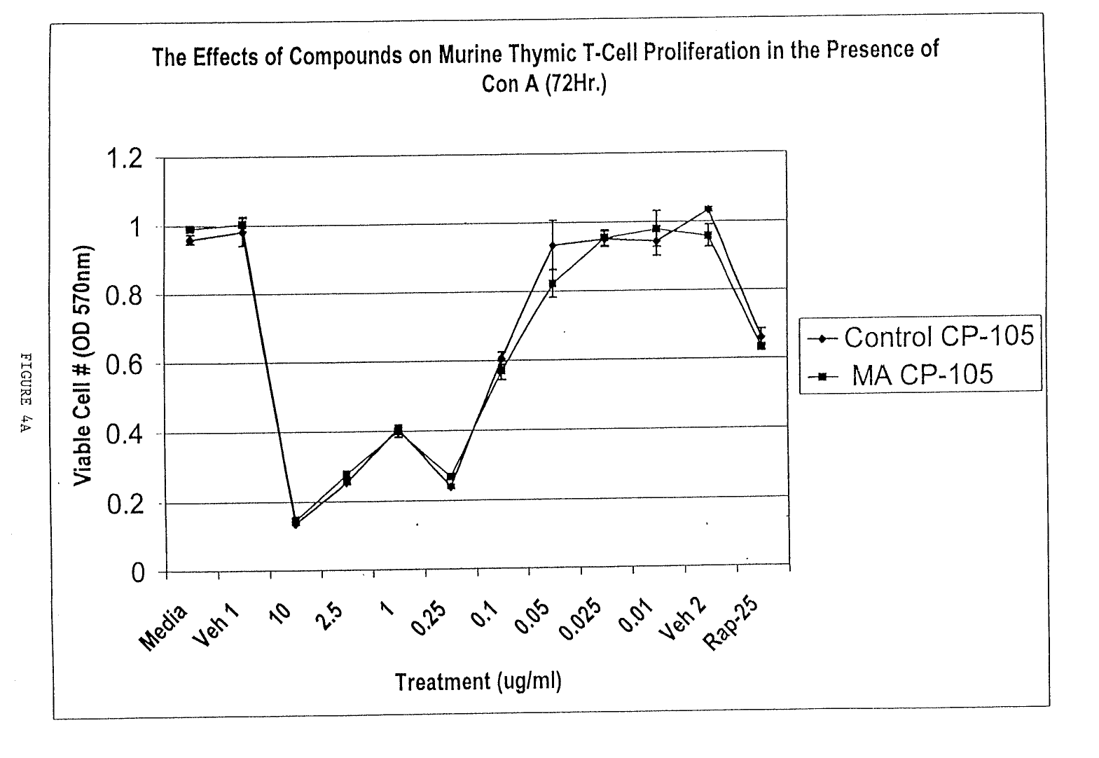

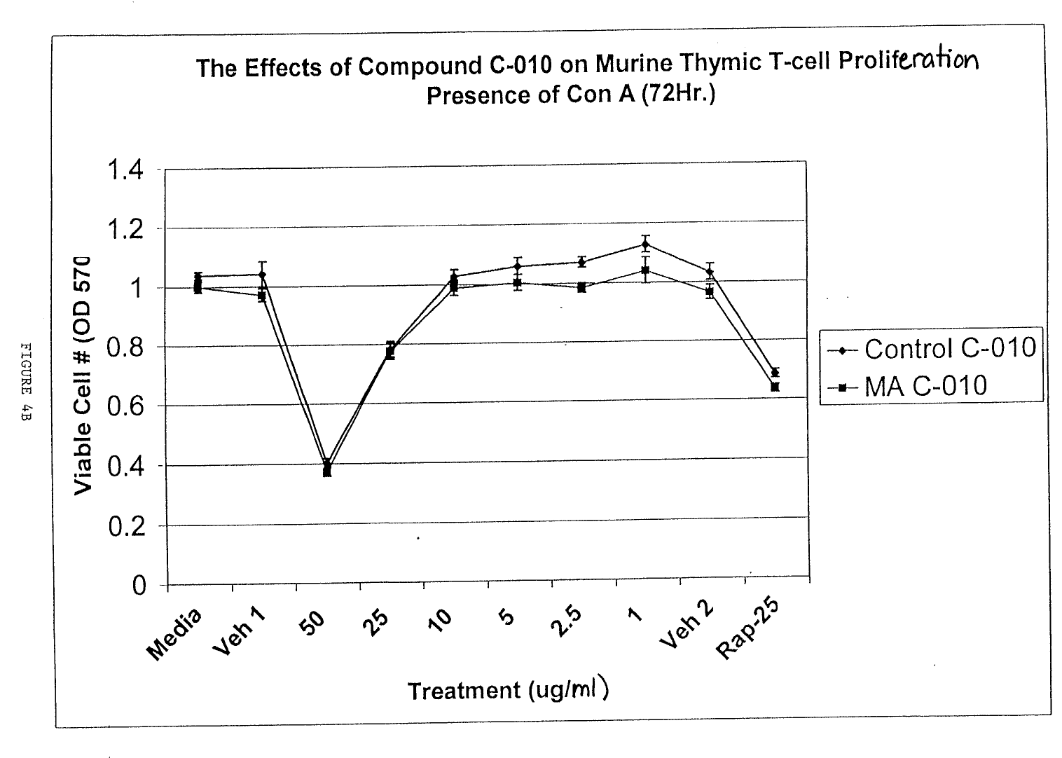

Murine thymic T cells were incubated with different concentrations of 25-hydroxycholesterol (CP 105; Panel A) or an oxysterol combined with lanolin (C-010; Panel B) in the absence or presence of 1 mM mevalonate (MA). Inhibition of T cell proliferation by 25-hydroxycholesterol, a known inhibitor of HMG-CoA reductase, or by an oxysterol combined with lanolin, C-0010-010, was not prevented by mevalonic acid (FIG. 4). This observation suggested that HMG-CoA reductase inhibitors may act synergistically with oxysterols to inhibit T cell proliferation and suppress inflammation.

### Example 5

### Synergistic Effect of HMG-CoA Reductase Inhibitors and Oxysterols

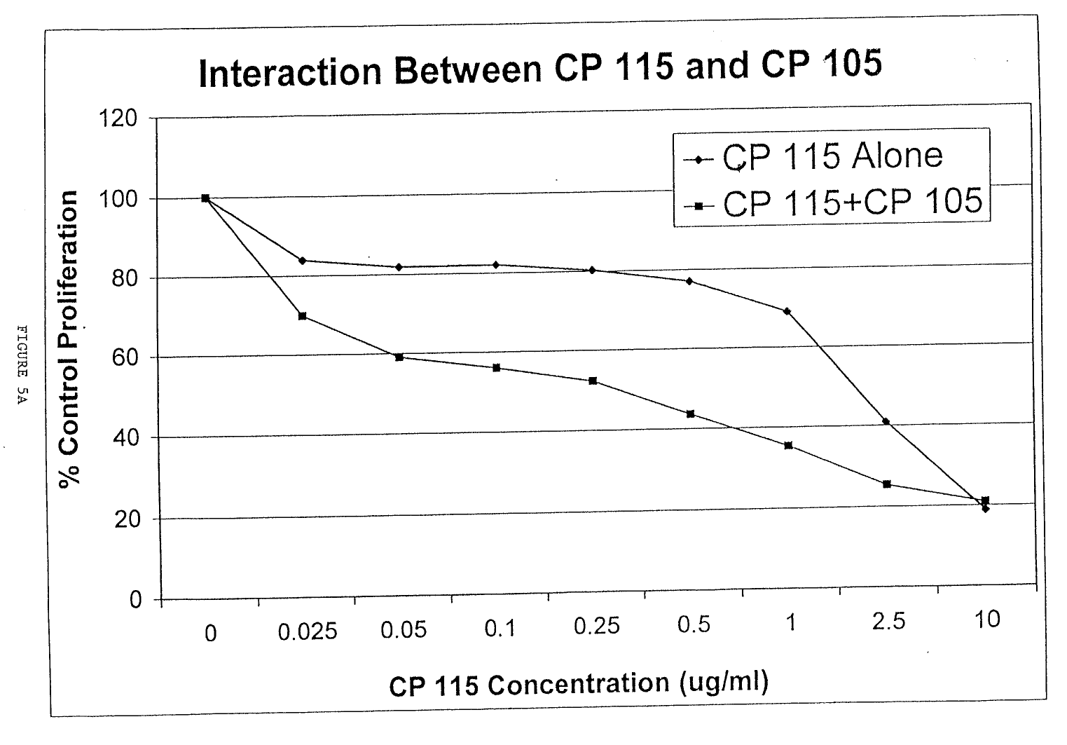

To further test this synergistic effect, T cells were incubated with different concentrations of mevastatin alone or in combination with 0.25 μg/ml 25-hydroxycholesterol (CP 105; FIG. 5, Panel A) or 10 μg/ml of an oxysterol combined with lanolin (C-010; FIG. 5, Panel B). The data was expressed as percent of control proliferation. CP 105 and C-010 inhibited T cell proliferation 14% and 28% at the indicated concentrations, respectively. Thus minimally effective concentrations of 25-hydroxycholesterol and C-0010-010 decreased the IC₅₀ of mevastatin by a factor greater than 10-fold (FIG. 5). This experiment confirmed that the inhibition of T cell proliferation by mevastatin is advantageously potentiated by oxysterols.

### Example 6

### Inhibition of IL-2 Gene Expression by Lovastatin

The effects of lovastatin on NFAT activation and IL-2 gene expression were investigated in Jurkat T cells (human leukemia T cells). Lovastatin inhibited the expression of an NFAT-regulated reporter gene in transiently transfected Jurkat cells stimulated with ionomycin and PMA or with thapsigargin + PMA. The effect of lovastatin was specific in that it also inhibited activation of the human IL-2 promoter, which contains several NFAT elements, but it did not affect transcription from a RSV-driven control reporter gene. Preincubation of Jurkat cells with mevalonic acid did not reverse the inhibition of NFAT reporter activity by lovastatin, suggesting that the compound acted through a mechanism unrelated to its known effects on HMG-CoA reductase and isoprenoid synthesis.

NFAT is synergistically activated by the Ca²⁺/calcineurin and PKC/ras/MAPK pathways. To gain further insight into the novel mechanism of action of lovastatin, dominant active forms of calcineurin and ras were expressed in Jurkat cells to bypass early signaling events mediated by ionomycin (i.e., Ca²⁺) and PMA (i.e., PKC). NFAT activity induced by ionomycin and dominant active ras was strongly inhibited by lovastatin, whereas the selective PKC inhibitor, Gö6850, was inactive, ruling out PKC as a target of lovastatin. In contrast, NFAT activity stimulated by PMA and dominant active calcineurin was relatively insensitive to lovastatin and inhibited by Gö6850. These results suggest that lovastatin acts upstream of calcineurin, i.e., most probably at the level of Ca²⁺ availability.

Since NFAT plays a critical role in IL-2 gene transcription, the effects of lovastatin on IL-2 mRNA levels and IL-2 secretion from stimulated lymphoid cells were investigated. Lovastatin inhibited both IL-2 mRNA accumulation and protein secretion in Jurkat cells stimulated with ionomycin and PMA. The inhibition of IL-2 secretion by lovastatin was dose-dependent, with an IC₅₀ of 12 μM (similar to that observed for inhibition of NFAT-dependent reporter activity), and was not reversed by addition of mevalonic acid. NFAT is also thought to regulate TNF-αgene expression, and lovastatin was found to inhibit TNF-αsecretion from ionomycin and PMA-stimulated Jurkat cells. These results demonstrated that lovastatin can exert effects on lymphocytes through a novel mechanism independent of its well known ability to inhibit HMG-CoA reductase and isoprenoid synthesis.

### Example 7

### Inhibition of Mast Cell Degranulation by Lovastatin

The RBL-2H3 mucosal mast cell line has been used extensively to study stimulus secretion coupling. Treatment with a calcium ionophore or antigenic crosslinking of the high affinity receptor for IgE on these cells leads to degranulation and the secretion of various proinflammatory mediators, including biogenic amines such as histamine and serotonin. Lovastatin inhibited the release of ³H-serotonin from RBL-2H3 cells stimulated with either antigen or calcium ionophore. Inhibition by lovastatin was dose-dependent and was not reversed by co-incubation with mevalonic acid. These results indicate that the inhibition of mast cell degranulation by lovastatin is not mediated through HMG-CoA reductase.

### Example 8

### Inhibition of T-cell Proliferation by an Inhibitor of Geranylgeranyl Transferase

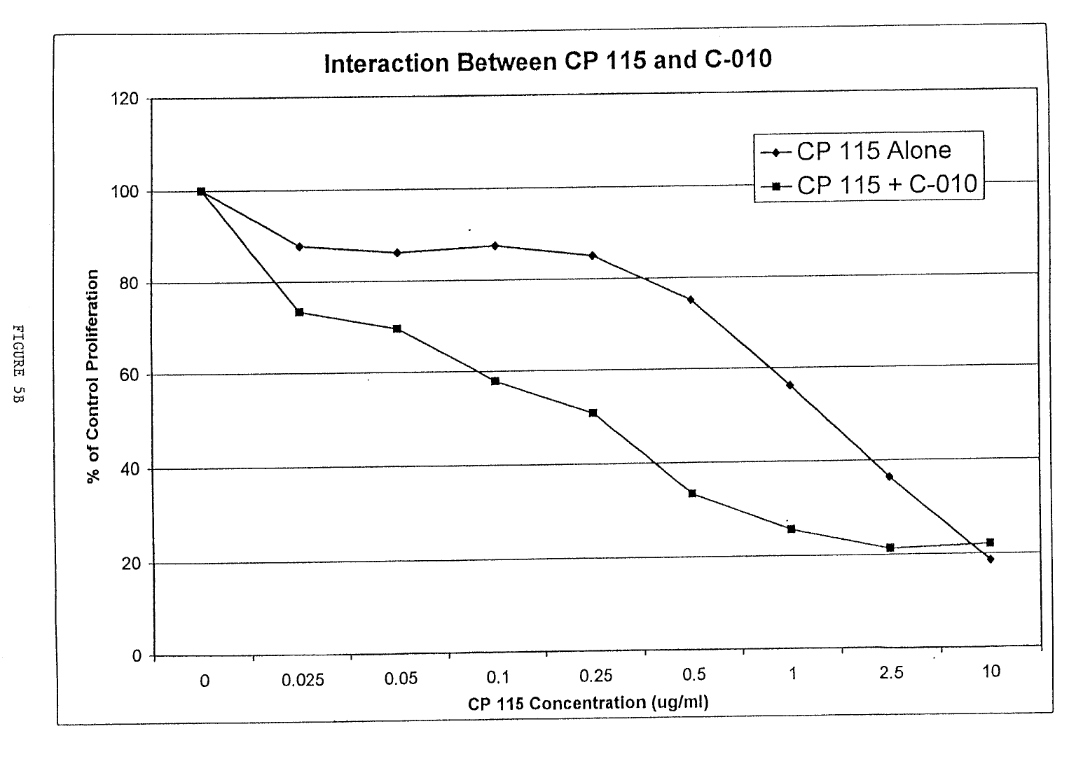

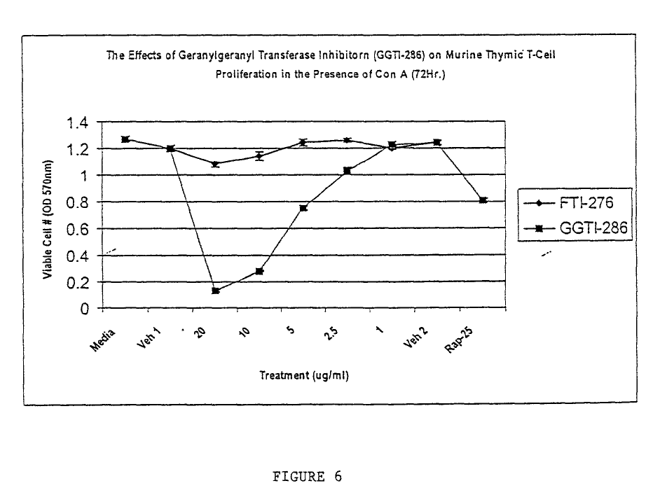

Murine thymic T cells were incubated with different concentrations of an inhibitor of geranylgeranyl transferase (GGTI). Dose-dependent inhibition of T cell proliferation by GGTI-286, a known inhibitor of geranylgeranyl transferase, were observed (FIG. 6). This observation demonstrated that inhibitors of geranylgeranyl transferase effectively block T cell proliferation and suppress inflammation.

### Example 9

### Animal Models for Inflammatory Conditions

1. Irritant Contact Dermatitis (ICD)

The phorbol ester 12-0-tetradecanoyl-13-phorbol acetate (TPA) is the active ingredient in cotton oil. TPA induced skin inflammation peaked between 24 and 30 hours when the ear was the target and between 8 and 16 hours when the flank was the target. These two alternative targets allowed to determine both the short and the longer term activity of different compounds in suppressing TPA-induced inflammation. The TPA swelling response is considered to be an acute irritant reaction mediated by a pro-inflammatory cytokine released from epidermal keratinocytes and infiltrating inflammatory cells that lacks an immune cell component.

Seven to eight week-old ICR female mice (SKH-1, Charles River or Hr/Hr, Simonsen Labs) or ICR Swiss albino female mice (Simonsen) were anaesthetized and treated with 5 μl of a TPA working solution on each ear surface. The TPA working solution was made from a 10 mg/ml stock solution in dimethylsulfoxide by a 1:40 dilution in ethanol to a final concentration of 0.4 nmol/μl. One hour after TPA treatment, the ears were treated with the anti-inflammatory agent. Ear thickness measurements were taken before TPA treatment and before anti-inflammatory agent application, as well as 6, 24 and 30 hours post-treatment, once the experiment was terminated. Ear swelling due to the acute irritant TPA reached a peak between 24 and 30 hours. Alternatively, this treatment was performed on the mouse flank using 25 μl of the same TPA concentration. In this case, swelling of the flank reached a peak between 8 and 16 hours, usually at 8 hours. It should be noted that a more severe reaction to TPA in terms of erythema and eventual necrosis of the outer pinna was observed in the hairless mouse population compared with that of the ICR mouse population.

To test the efficacy of a potential anti-inflammatory agent, the agent was incorporated into the solution containing TPA, with or without excipients such as safflower oil, or avocado oil, and applied to the mouse ear with the TPA irritant. The effect of the anti-inflammatory drug was measured by the ear swelling response as described above.

To test an anti-inflammatory agent in transdermal patch applications, the agent was incorporated into the adhesive before casting the adhesive onto either the release liner, semi-permeable membrane or the backing material of a conventional transdermal patch. Alternatively, an anti-inflammatory agent was incorporated into the drug reservoir and applied to the skin concurrently. The irritation or allergic reactions due to either the drug, the adhesive used, the penetration enhancers and solvents were mitigated by the anti-inflammatory agent incorporated into the patch. In this approach, TPA was used as an irritant drug model and the effect of the anti-inflammatory agent was measured as the swelling response induced by the patch to the mouse skin.

2. Allergic Contact Dermatitis (ACD)

Allergic contact hypersensitivity (ACH) is a clinically important type of dermatitis that can occur as a result of exposure to occupational or environmental agents. A number of chemicals can be used to experimentally reproduce this phenomenon. The sequence of events following initial application of a contact allergen to the skin (sensitization phase) is thought to involve presentation of the allergen in association with MHC class II molecules by the Langerhans cells (LCs). LCs migrate to the regional lymph nodes where they stimulate antigen-specific T cell proliferation. When the allergen is reapplied to the skin several days later (challenge phase), an allergic reaction occurs that takes 24-48 hours to develop. This type of response is termed delayed type hypersensitivity, in contrast to the immediate hypersensitivity mediated by mast cell degranulation.

Challenging mouse ears with haptens, such as 1-chloro-2,4-dinitrobenzene (DNCB), 2,4-dinitrofluorobenzene (DNFB), or oxazolone after a 5 day sensitization phase, induced severe swelling associated with an immune response that included a memory T cell infiltrate. Maximum swelling occurred between 24 and 48 hours after challenge. Thus, DNFB, DCNB and oxazolone were considered to induce delayed contact hypersensitivity.

The protocol involved shaving the posterior portion of the backs of anesthetized 7-8 week old female Balb/c mice with electric clippers. DNCB in acetone:olive oil (4:1), DNFB in acetone alone or oxazolone (15% oxazolone in 97% acetone/3% DMSO) was applied to the shaved area in a volume of 50 μl (DNCB), 100 μl (DNFB), or 20 μl (oxazolone). The concentration of DNCB in the sensitizing solution ranged from 0.5% to 4% w/v or 0.1 to 0.5% for DNFB. Five days after sensitization, the thickness of both ears was measured with a spring-loaded micrometer and both surfaces of the ears were challenged with either 10 μl of DNCB in acetone:olive oil ranging in concentration from 0.25%-2.5% w/v, 10 μl of DNFB in acetone at 0.1%, 10 μl 2% oxazolone in 97% acetone/3% DMSO, or vehicle alone. Post challenge ear thickness measurements were taken at 8, 16, 24, 32, 48, 56 and 72 hours. Typical ear thickness measurements were more variable than in the TPA model and depended on the sensitization and challenge doses of hapten as well on the individual groups of mice. However, a swelling response of at least 50% above baseline was considered reasonable. Although it was assumed that only the challenged ears demonstrated swelling, the control ears were used to identify any effects of sensitization and the vehicle on inflammation.

### Example 10

### Inhibition of TPA-Induced Acute Inflammation in Mice with Mevastatin

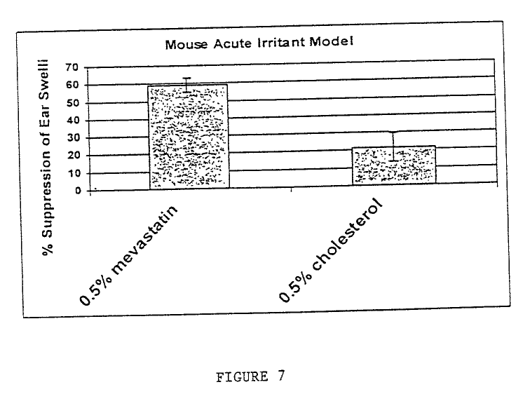

These experiments demonstrate the ability of HMG-CoA reductase inhibitors, e.g., mevastatin, to reduce acute inflammation induced by topical TPA challenge. The model systems described in Example 9 were employed, using TPA challenge as a model for acute inflammation. Mevastatin formulations (0.5% in an vehicle of ethanol/cyclohexane (1:1)) were applied to mice that had been treated with TPA, either 30 minutes or one hour after challenge. The percent suppression for each formulation was determined as described in Example 9 at 30 hours post-challenge (for TPA). Results are reported as the percent suppression of inflammation compared to untreated controls, as measured by ear thickness. Surprisingly, as little as 0.5% mevastatin reduced TPA-induced acute inflammation by 58% (FIG. 7).

### Example 11

### Inhibition of DNFB-Induced Contact Hypersensitivity Reactions in Mice with Topical Simvastatin-, Lovastatin- and Oxysterol-Containing Formulations

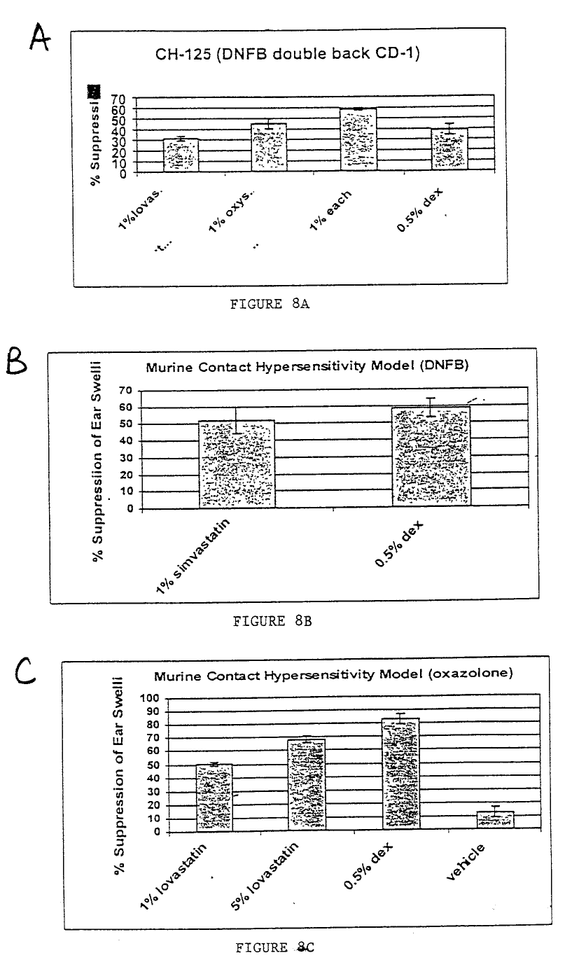

These experiments demonstrate that formulations containing HMG-CoA reductase inhibitors such as lovastatin, simvastatin and an oxysterol are also able to significantly reduce delayed contact hypersensitivity inflammation. As in Example 9, DNFB challenge was used as a model for delayed contact hypersensitivity. A lovastatin formulation (1%), an oxysterol-containing formulation(1%) or the combination of the two (1% each) were applied to mice that had been treated with DNFB, either 30 minutes or one hour after challenge. The percent suppression for each formulation was determined at 48 hours post-challenge. Results are reported as the percent suppression of inflammation compared to untreated controls, as measured by ear thickness. 1% lovastatin in a vehicle (ethanol/cyclohexane/DMSO (75:22:3)) reduced delayed contact hypersensitivity induced by DNFB (FIG. 8A). Similar results were observed with 1% simvastatin (FIG. 8B). The combination of a 1% lovastatin formulation and a 1% oxysterol combined with lanolin was a surprisingly effective formulation in suppressing ear swelling response induced by DNFB and the results obtained exceeded those obtained with either lovastatin or an oxysterol-containing formulation alone. The effect observed with lovastatin was dose-dependent in a similar model where the topical sensitizer was oxazolone instead of DNFB.

### Example 12

### Topical Formulations Containing Lovastatin

Lovastatin can be prepared in pharmaceutically acceptable dosage forms, especially topical dosage forms, such as, e.g., creams, lotions and gels. Two topical dosage forms containing lovastatin are illustrated in Table 1, below.

TABLE 1

| Compound | Weight | |
|---|---|---|
| Lovastatin | 0.1 | g |
| | to 10 | g |
| Ethanol | 30 | g |
| Carbomer 1382 | 1 | g |
| Propylene glycol | 30 | g |
| Oleic acid | 1 | g |
| Cholesterol | 1 | g |
| Water | q.s. | |
| Total | 100 | g |
| Lovastatin | 0.1 | g |
| | to 10 | g |
| Propylene glycol | 5 | g |
| Brij 58 | 2.5 | g |
| Cetostearyl alcohol | 5 | g |
| Immidurea | 0.5 | g |
| Cholesterol | 1 | g |
| Isopropyl Myristate | 5 | g |

| | |
|---|---|
| Water | q.s. |

### Example 13

### Method of Treating Patients with Atopic Dermatitis with Lovastatin

Patients with clinically defined atopic dermatitis were selected and provided with the lovastatin cream. The areas of the skin afflicted with erythema, induration, excoriation, lichenification and pruritis were treated with the lovastatin cream either once or twice every day until the symptoms of the disease dissipated. Patients 10 using systemic glucocorticoids or immunosuppressants (e.g., Tacrolimus or cyclosporin) were allowed to use the topical lovastatin cream concomitantly with the therapy or upon completion of the therapy. Patients using glucocorticoids or immunosuppressants (e.g., Tacrolimus; Protopic) topically once per day were treated with the lovastatin cream at a different time during the day to accelerate the recovery from disease and potentially minimize the use of the topical glucocorticoids. Once the patients completed an initial course of topical glucocorticoid or immunosuppressant treatment, patients started using a topical lovastatin cream to prevent relapse of the disease.

### Example 14

### Method of Treating of Asthma with Aerosolized Lovastatin

Asthma patients with wheezing and/or breathing difficulties were treated with lovastatin in an aerosolized form or as a nasal inhaler to allow direct delivery of the lovastatin to the lung tissue. The lovastatin can be formulated alone or in conjunction with a beta-adrenergic agonist (e.g., albuterol) or a glucocorticoid (e.g., beclomethasone diproprionate). The use of lovastatin provided more effective relief of lung constriction and inflammation and minimized the use of beta-agonists or glucocorticoids, allowing to reduce the severity of the side effects associated with either therapy.

### Example 15

### Method of Treating of Patients Afflicted with Atopic Dermatitis (AD) with Lovastatin

Atopic dermatitis is a chronically relapsing, pruritic inflammatory skin disorder. While severe atopic dermatitis is characterized by severe exudative papules, intense pruritus and erythema lichenification and excoriation, mild-to-moderate atopic dermatitis is typically manifested as red patches of dry skin with excoriation, infiltration and papulation. Patients diagnosed with atopic dermatitis are to apply the lovastatin cream to the afflicted area(s), either twice or three times daily. The overall improvement of AD can be observed as early as two to three weeks following the treatment. In one embodiment, the improvement can be evaluated using an Eczema Area Severity Index (EASI) score (see, e.g., Hanifin, J M et aL, Current Therapeutic Research, 59(4):227-233; Paller, A et aL, J Am. Acad. Dermatol., 44(1):S47-57 (2001); and Hanifin, J M, et al., J Am. Acad. Dermatol., 44(1):S28-38 (2001), the teachings of all of which are incorporated herein by reference), wherein a reduction of 20-25% of the disease severity is considered an improvement. Lovastatin cream can also be used as maintenance therapy to prevent the relapse of AD, as follow-up therapy to a two-week course of steroid therapy, and as combination therapy to reduce the steroid dose. This safer and more effective therapy is extremely useful in the pediatric population where steroid use can lead to growth delays and other debilitating steroid-induced side effects.

What is claimed is:

1. A method for treating a skin disorder, wherein the skin disorder is selected from the group consisting of atopic dermatitis and skin photodamage, the method comprising administering to a patient in need thereof an HMG-CoA reductase inhibitor in a therapeutically effective amount.

2. The method of claim 1, wherein said HMG-CoA reductase inhibitor is selected from the group consisting of mevastatin, lovastatin, fluvastatin, pravastatin, simvastatin, dalvastatin, cerivastatin and atorvastatin.

3. The method of claim 1, wherein said HMG-CoA reductase inhibitor is an oxysterol.

4. The method of claim 3, wherein said oxysterol is 25-hydroxycholesterol.

5. The method of claim 1, wherein said HMG-CoA reductase inhibitor is administered topically.

6. The method of claim 1, wherein said HMG-CoA reductase inhibitor is formulated in a pharmaceutical composition.

7. A method for treating a skin disorder, wherein the skin disorder is selected from the group consisting of atopic dermatitis, skin photodamage, extrinsic skin aging, skin photoaging and skin irritation, the method comprising administering to a patient in need thereof a protein prenylation inhibitor in a therapeutically effective amount.

8. The method of claim 7, wherein said protein prenylation inhibitor is a peptide analog.

9. The method of claim 8, wherein said peptide analog is selected from the group consisting of GGTI-286 and FTI-276.

10. The method of claim 7, wherein said protein prenylation inhibitor is a monoterpene.

11. The method of claim 10, wherein said monoterpene is selected from the group consisting of d-limonene, perillic acid and perillyl alcohol.

12. The method of claim 7, wherein said protein prenylation inhibitor is administered topically.

13. The method of claim 7, wherein said protein prenylation inhibitor is formulated in a pharmaceutical composition.

14. A method for treating skin inflammation, the method comprising administering to a patient in need thereof a protein prenylation inhibitor in a therapeutically effective amount.

15. The method of claim 14, wherein said protein prenylation inhibitor is a peptide analog.

16. The method of claim 15, wherein said peptide analog is selected from the group consisting of GGTI-286 and FTI-276.

17. The method of claim 14, wherein said protein prenylation inhibitor is a monoterpene.

18. The method of claim 17, wherein said monoterpene is selected from the group consisting of d-limonene, perillic acid and perillyl alcohol.

19. The method of claim 14, wherein said protein prenylation inhibitor is administered topically.

20. The method of claim 14, wherein said protein prenylation inhibitor is formulated in a pharmaceutical composition.

21. A pharmaceutical composition comprising a protein prenylation inhibitor and a topical carrier.

22. The pharmaceutical composition of claim 21, wherein said protein prenylation inhibitor is a peptide analog selected from the group consisting of GGTI-286 and FTI-276.

23. The pharmaceutical composition of claim 21, wherein said protein prenylation inhibitor is a monoterpene selected from the group consisting of d-limonene, perillic acid and perillyl alcohol.

24. A method for treating a skin disorder, wherein the skin disorder is selected from the group consisting of inflammatory skin disease, atopic dermatitis, skin photodamage, extrinsic skin aging, skin photoaging and skin irritation, the method comprising administering to a patient in need thereof a combination comprising at least two inhibitors of cholesterol biosynthesis in a therapeutically effective amount, wherein the first inhibitor is an HMG-CoA reductase inhibitor.

25. The method of claim 24, wherein the second inhibitor is an inhibitor of the downstream mevalonate metabolism.

26. The method of claim 24, wherein said cholesterol biosynthesis inhibitors are administered simultaneously.

27. The method of claim 24, wherein said cholesterol biosynthesis inhibitors are administered topically.

28. The method of claim 24, wherein said HMG-CoA reductase inhibitor is selected from the group consisting of mevastatin, lovastatin, fluvastatin, pravastatin, simvastatin, dalvastatin, cerivastatin and atorvastatin.

29. The method of claim 24, wherein said HMG-CoA reductase inhibitor is an oxysterol.

30. The method of claim 29, wherein said oxysterol is 25-hydroxycholesterol.

31. A method for treating a skin disorder, wherein the skin disorder is selected from the group consisting of hyperproliferative skin disorder and psoriasis, the method comprising administering to a patient in need thereof a combination comprising an HMG-CoA reductase inhibitor and an inhibitor of the downstream mevalonate metabolism.

32. The method of claim 31 ,wherein said HMG-CoA reductase inhibitor and said downstream mevalonate metabolism inhibitor are administered simultaneously.

33. The method of claim 31, wherein said HMG-CoA reductase inhibitor and said downstream mevalonate metabolism inhibitor are administered topically.

34. The method of claim 31, wherein said HMG-CoA reductase inhibitor is selected from the group consisting of mevastatin, lovastatin, fluvastatin, pravastatin, simvastatin, dalvastatin, cerivastatin and atorvastatin.

35. The method of claim 31, wherein said HMG-CoA reductase inhibitor is an oxysterol.

36. The method of claim 35, wherein said oxysterol is 25-hydroxycholesterol.

37. The method of claim 31, wherein said HMG-CoA reductase inhibitor and said downstream mevalonate metabolism inhibitor are formulated in a pharmaceutical composition.

38. A method for enhancing the potency of an anti-inflammatory drug, said method comprising administering to a patient in need thereof said anti-inflammatory drug and an excipient comprising an HMG-CoA reductase inhibitor.

39. The method of claim 38, wherein said HMG-CoA reductase inhibitor is selected from the group consisting of mevastatin, lovastatin, fluvastatin, pravastatin, simvastatin, dalvastatin, cerivastatin and atorvastatin.

40. The method of claim 38, wherein said HMG-CoA reductase inhibitor is an oxysterol.

41. The method of claim 40, wherein said oxysterol is 25-hydroxycholesterol.

42. The method of claim 40, wherein said anti-inflammatory drug is selected from the group consisting of corticosteroids, salicylates, colchicine, para-aminophenol, propionic acid, piroxicam, ketorolac, ketoprofen, cyclooxygenase type II inhibitors and indomethacin.

43. A method for suppressing mucocutaneous inflammation and irritation caused by transdermal or transmucosal drug delivery, irritating drug delivery enhancers or irritating drug substances, said method comprising administering to a patient in need thereof a therapeutic compound in combination with an excipient comprising an HMG-CoA reductase inhibitor.

44. The method of claim 43, wherein said HMG-CoA reductase inhibitor is selected from the group consisting of mevastatin, lovastatin, fluvastatin, pravastatin, simvastatin, dalvastatin, cerivastatin and atorvastatin.

45. The method of claim 43, wherein said HMG-CoA reductase inhibitor is an oxysterol.

46. The method of claim 43, wherein said oxysterol is 25-hydroxycholesterol.

47. The method of claim 43, wherein said therapeutic compound is selected from the group consisting of glycerol, corticosteroids and salicylates.
